(19) United States
(12) Patent Application Publication
(10) Pub. No.: US 2016/0151262 A1
(43) Pub. Date: Jun. 2, 2016

(54) Gelling Agents For Use In Cosmetic Compositions

(71) Applicants: Wei Yu, Edison, NJ (US); Vijay Joshi, Livingston, NJ (US)

(72) Inventors: Wei Yu, Edison, NJ (US); Vijay Joshi, Livingston, NJ (US)

(21) Appl. No.: 14/457,622

(22) Filed: Aug. 12, 2014

(51) Int. Cl. A61K 8/40; A61K 8/02

(52) U.S. Cl. CPC A61K 8/40; A61K 8/0233; A61K 8/0229; A61Q 15/00

(57) ABSTRACT

The present invention is directed to a composition comprising a gelling agent and an at least one oil, wherein the oil and gelling agent are in a ratio of about 2 to about 16.

This application claims priority from copending international application Serial Number PCT/US2013/026334, filed Feb. 15, 2013, which claims priority to U.S. provisional application 61/600,087, filed Feb. 17, 2012, now expired, the entire disclosure of both applications is hereby incorporated by reference.

### FIELD OF THE INVENTION

The present invention generally relates to a novel composition which is highly wearable transfer-resistant with excellent long wearing properties which, until now, could only be achieved through the use of traditional gelling agents, such as, natural and synthetic waxes. Moreover, the present invention relates to a composition that is easily and uniformly deposited and spread onto a keratinous substrate, having a unique texture and feel.

### BACKGROUND OF THE INVENTION

Many compositions, especially cosmetic compositions, have been developed for easy and comfortable application onto a targeted substrate. Unfortunately, many of these compositions are in fact difficult to apply and do not possess a smooth feel upon application. Moreover, compositions often times have the undesirable characteristics of stickiness, poor flexibility, and hardness (too soft or too hard). In addition traditional gelling agents do not provide a clear base which is desirable for cosmetic compositions. The undesirable characteristics and lack of clarity can be linked to the use of traditional gelling agents.

Therefore, it is an object of the present invention to provide a composition capable of possessing a unique creamy texture and feel, having exceptional application properties, even in solid form, while at the same time being long wearing and transfer resistant without having to use traditional gelling agents, and which can serve as a stable clear base/matrix for the incorporation of various types of ingredients.

Cosmetic compositions have been formulated substituting polyamides for natural or synthetic waxes but this substitution does not yield the unique creamy texture and feel, or a clear base, having exceptional application properties sought in a cosmetic composition.

It is the object of this invention to provide a stable clear base/matrix that provides the unique creamy texture and feel and having exceptional application properties using a minimum amount of ingredients.

### BRIEF SUMMARY OF THE INVENTION

In an embodiment the present invention is directed to a composition comprising a gelling agent and an at least one oil, wherein the oil and gelling agent are in a ratio of about 2 to about 16. The best oil to gelling agent ratio is about 4 to about 10.

In another embodiment the gelling agent(s) is a compound having a structure of (I):

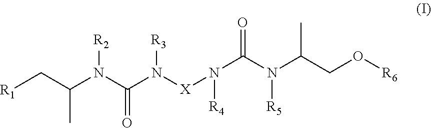

R₂, R₃, R₄, R₅, which are the same or different are selected from a hydrogen, a C₁-C₂₄ alkyl group;
X is selected from a heteroatom, C₂ to C₂₄ alkyl;
R₁ and R₆ which are the same or different are selected from a hydrogen, a C₁-C₂₄ alkyl group, a heteroatom.

In an embodiment the preferred gelling agent is 1-[1-Methyl-2-(1-methyl-2-tridecyloxy-ethoxy)-ethyl]-3-(6-{3-[1-methyl-2-(1-methyl-2-tridecyloxy-ethoxy)-ethyl]-ureido}-hexyl)-urea.

In an embodiment the above-identified gelling agent is present in a concentration of about 6 to 14 weight percent of the composition

In an embodiment the preferred composition of the present invention is a cosmetic composition. In another embodiment the composition can be anhydrous or an emulsion. When the composition is an emulsion the water phase and oil phase have a matched refractive index. The composition has a base comprised of a combination of gelling agent and oil. Additional ingredients can be added to the base and include for example, at least one additive chosen from an antioxidant, a filler, a film-former, a preserving agent, a fragrance, a neutralizing agent, a thickener, a cosmetic composition active agent, an emollient, a solvent, a pigment, a dye, or a stain and mixtures thereof.

In an embodiment the combination of the oil and gelling agent forms a clear gel which is used as a base composition to which other ingredients are added. The composition can take the form of a stick, for example, a lipstick. The stick can be a uniform color or can have a pigmented core surrounded by a clear shell.

In an embodiment the composition using the oil and gelling agents disclosed herein has a flexibility of >about 30%, a stickiness of <about −10 grams, and a hardness of <about 100 grams.

### DETAILED DESCRIPTION OF THE DRAWINGS

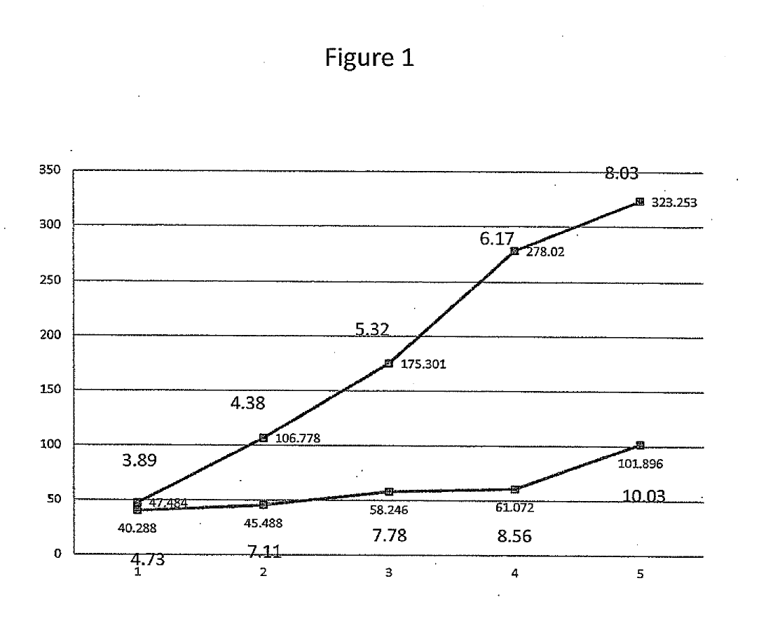

FIG. 1 compares hardness of the gelling agent and oil combination found in Example 1 and Example 2. The points on the line are the various ratios of oil/gallant. The y axis is the hardness value.

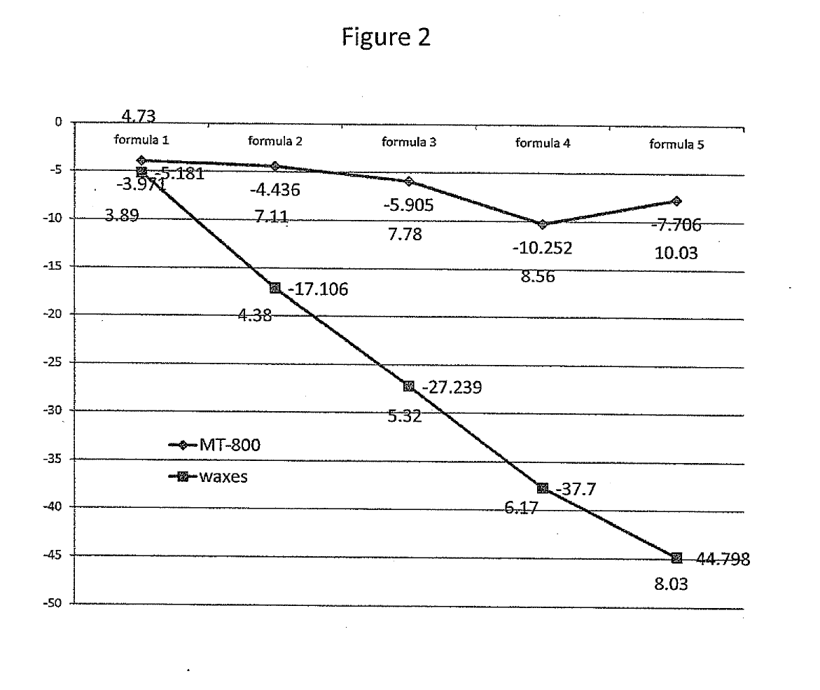

FIG. 2 compares stickiness of the gelling agent and oil combination found in Example 1 and Example 2. The points on the line are the various ratios of oil/gallant. The y axis is the stickiness value.

### DETAILED DESCRIPTION OF THE INVENTION

The present invention is directed to a composition comprising a gelling agent and an at least one oil, wherein the oil and gelling agent are in a ratio of about 2 to about 16. The best oil to gelling agent ratio is about 4 to about 10.

The terms used in this specification generally have their ordinary meanings in the art, within the context of the invention, and in the specific context where each term is used. Certain terms are discussed below, or elsewhere in the specification, to provide additional guidance to the practitioner in describing the compounds, compositions, and methods of the invention and how to make and use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. The use of examples anywhere in this specification, including examples of any terms discussed herein, is illustrative only, and in no way limits the scope and meaning of the invention or of any exemplified term. Likewise, the invention is not limited to the examples presented.

As used herein, “about” or “approximately” shall generally mean within 20 percent, preferably within 10 percent, and more preferably within 5 percent of a given value or range. Other than in the operating examples, or where otherwise indicated, all numbers expressing quantities of ingredients and/or reaction conditions are to be understood as being modified in all instances by the term “about”.

“Film former” or “film forming agent” or “film forming resin” as used herein means a polymer which, after dissolution in at least one solvent (such as, for example, water and organic solvents), leaves a film on the substrate to which it is applied, for example, once the at least one solvent evaporates, absorbs and/or dissipates on the substrate.

“Keratinous substrates”, as used herein, include but are not limited to, skin, hair and nails.

As used herein, the term ratio is used to express the relationship in quantity or amount of the oil and gelling agent.

As used herein, the term anhydrous means a composition has little or no water. An anhydrous composition is a composition having <1% water.

As used herein, the expression “at least one” means one or more and thus includes individual components as well as mixtures/combinations.

“Long wear” compositions as used herein, refers to compositions where color remains the same or substantially the same as at the time of application, as viewed by the naked eye, after an extended period of time. Long wear properties may be evaluated by any method known in the art for evaluating such properties. For example, long wear may be evaluated by a test involving the application of a composition to human hair, skin or lips and evaluating the color of the composition after an extended period of time. For example, the color of a composition may be evaluated immediately following application to hair, skin or lips and these characteristics may then be re-evaluated and compared after a certain amount of time. Further, these characteristics may be evaluated with respect to other compositions, such as commercially available compositions.

“Transfer resistance” or resistance as used herein refers to the quality exhibited by compositions that are not readily removed by contact with another material, such as, for example, a glass, an item of clothing or the skin, for example, when eating or drinking. Transfer resistance may be evaluated by any method known in the art for evaluating such. For example, transfer resistance of a composition may be evaluated by a “kiss” test. The “kiss” test may involve application of the composition to human keratin material such as hair, skin or lips followed by rubbing a material, for example, a sheet of paper, against the hair, skin or lips after expiration of a certain amount of time following application, such as 2 minutes after application. Similarly, transfer resistance of a composition may be evaluated by the amount of product transferred from a wearer to any other substrate, such as transfer from the hair, skin or lips of an individual to a collar when putting on clothing after the expiration of a certain amount of time following application of the composition to the hair, skin or lips. The amount of composition transferred to the substrate (e.g., collar, or paper) may then be evaluated and compared. For example, a composition may be transfer resistant if a majority of the product is left on the wearer's hair, skin or lips. Further, the amount transferred may be compared with that transferred by other compositions, such as commercially available compositions. In a preferred embodiment of the present invention, little or no composition is transferred to the substrate from the hair, skin or lips.

In one embodiment, the composition may also comprise at least one coloring agent chosen from pigments and dyes or a combination of pigments and dyes. As used herein, pigments refer to colored solid particles at 25° C. that are not soluble in the liquid fatty phase. Pigments may include nacreous pigments (i.e., nacres), and pearling agents.

The at least one coloring agent may be chosen, for example, in order to obtain make-up compositions which give good coverage, in other words, which do not leave a significant amount of the at least one keratin material to which it is applied showing through. The coloring agent may also reduce the sticky feel of the compositions.

Representative oil soluble dyes which may be used include, but are not limited to, Sudan red, DC Red 17, DC Green 6, β-carotene, soybean oil, Sudan brown, DC Yellow 11, DC Violet 2, DC Orange 5, annatto, and quinoline yellow and natural oil soluble dyes. The liposoluble dyes, when present, may have a concentration ranging up to 20% by weight of the total weight of the composition, such as from 0.01% to 6%.

Representative water soluble dyes which may be used include, but are not limited to DC red 33, red 22, red 28, blue 1, yellow 5, yellow 6, red 40, yellow 10, green 3, green 5, green 8 and natural water soluble dyes. The water soluble dyes, when present, may have a concentration ranging up to 20% by weight of the total weight of the composition, such as from 0.01% to 6%.

In one aspect, the pigments may be chosen from white, colored, mineral, organic, coated and uncoated pigments. Representative examples of mineral pigments include, but are not limited to, titanium dioxide, which may be optionally surface-treated, zirconium oxide, zinc oxide, cerium oxide, iron oxides, chromium oxides, manganese violet, ultramarine blue, chromium hydrate and ferric blue. Representative examples of organic pigments include, but are not limited to, carbon black, pigments of D & C type, and lakes based on cochineal carmine, barium, strontium, calcium and aluminum. If present, the pigments may have a concentration ranging up to 40% by weight of the total weight of the composition, and for example from 1% to 35%, and further such as from 2% to 25%. In one embodiment comprising a face powder product, the pigments, including nacreous pigments, may, for example, represent up to 50% by weight of the composition.

In one aspect, the nacreous pigments (or nacres) may be chosen from white nacreous pigments such as mica coated with titanium or with bismuth oxychloride; colored nacreous pigments such as titanium mica with iron oxides, titanium mica with ferric blue or chromium oxide, and titanium mica with an organic pigment chosen from those mentioned above; and nacreous pigments based on bismuth oxychloride. The nacres, if present, may have a concentration ranging up to 30% by weight of the total weight of the composition, such as from 0.1% to 20%. silicone chain, and wherein the groups each comprise from 2 to 24 carbon atoms; phenylsilicones such as phenyl trimethicones, phenyl dimethicones, phenyl trimethylsiloxy diphenylsiloxanes, diphenyl dimethicones, diphenyl methyldiphenyl trisiloxanes and 2-phenylethyl trimethylsiloxysilicates; hydrocarbons chosen from linear and branched, volatile and non-volatile hydrocarbons of synthetic and mineral origin, such as volatile liquid paraffins (such as isoparaffins and isododecane) or non-volatile liquid paraffins and derivatives thereof; and liquid petrolatum, liquid lanolin, polydecenes, hydrogenated polyisobutene such as hydrogenated polybutene, e.g., PARLEAM from Nippon Oil Fats and squalane.

In one embodiment, the cosmetic composition is non-tinted, e.g., in a form of a clear lipstick or antiperspirant/deodorant. In another embodiment, the cosmetic composition is tinted and comprises a coloring agent chosen from pigments and dyes.

As used herein, pigments refer to colored solid particles at 25° C. that are not soluble in the liquid fatty phase. Pigments may include nacreous pigments (i.e., nacres), and pearling agents.

Representative oil soluble dyes which may be used include, but are not limited to, Sudan red, DC Red 17, DC Green 6, β-carotene, soybean oil, Sudan brown, DC Yellow 11, DC Violet 2, DC Orange 5, annatto, and quinoline yellow and natural oil soluble dyes. The liposoluble dyes, when present, may have a concentration ranging up to 20% by weight of the total weight of the composition, such as from 0.01% to 6%.

Representative water soluble dyes which may be used include, but are not limited to DC red 33, red 22, red 28, blue 1, yellow 5, yellow 6, red 40, yellow 10, green 3, green 5, green 8 and natural water soluble dyes. The water soluble dyes, when present, may have a concentration ranging up to 20% by weight of the total weight of the composition, such as from 0.01% to 6%.

For the purposes of the present invention, the use of the term “cosmetic composition” is understood to mean a composition suitable for application to the human body. A cosmetic composition is typically applied to the body for beautifying, cleansing, moisturizing or otherwise treating the external surface of the body, including by cleansing, coloring, conditioning, or protecting the external surface of the body part such as, for example, the skin, nails, lips, or hair. Examples of cosmetic compositions in which the present compounds can be used include a mascara, lipstick, lip-gloss, lip-balm, and antiperspirant/deodorant.

For purposes of this invention a gelling agent is limited to compounds having the following structure (I):

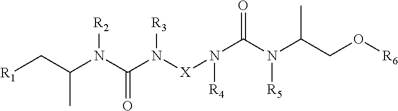

R₂, R₃ R₄, R₅, which are the same or different are selected from a hydrogen, a C₁-C₄ alkyl group;

X, is selected from a heteroatom, C₂ to C₂₄ alkyl;

R₁ and R₆ which are the same or different are selected from a hydrogen, a C₁-C₂₄ alkyl group, a heteroatom.

An example of a gelling agent includes but is not limited to 1-[1-Methyl-2-(1-methyl-2-tridecyloxy-ethoxy)-ethyl]-3-(6-{3-[1-methyl-2-(1-methyl-2-tridecyloxy-ethoxy)-ethyl]-ureido}-hexyl)-urea which is known as MT800.

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. In an embodiment, a straight chain or branched chain alkyl has 24 or fewer carbon atoms in its backbone. The term “alkyl” can be used alone or as part of a chemical name as in for example, “trialkylorthoformate”. The alkyl portion of the alkyl, alkoxy, alkanoyloxy, alkoxymethyl, alkanoyloxymethyl, alkylsulphinyl, alkylsulphonyl, alkylsulfonamido, carboalkoxy, carboalkyl, alkanoylamino aminoalkyl, alkylaminoalkyl, N,N-dicycloalkylaminoalkyl, hydroxyalkyl, and alkoxyalkyl substituents include both straight chain as well as branched carbon chains. Moreover, the term “alkyl” as used throughout the specification and claims is intended to include both “unsubstituted alkyls” and “substituted alkyls”, the latter of which refers to alkyl moieties having substituents replacing a hydrogen on one or more carbons of the hydrocarbon backbone. Such substituents can include, for example, a halogen, a hydroxyl, a carbonyl (such as a carboxyl, an ester, a formyl, or a ketone), a thiocarbonyl (such as a thioester, a thioacetate, or a thioformate), an alkoxyl, a phosphoryl, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF, —CN and the like. For purposes of this disclosure carbon carbon bonds may be single, double, or triple bonds.

The term “heteroatom” as used herein means an atom of any element other than carbon or hydrogen and include for example nitrogen, oxygen, sulfur, phosporus, and selenium.

As used herein, the term oil refers to an ester, a volatile solvent, a non-volatile oil, and a volatile oil. The preferred oil(s) used are oils that when combined with the gelling agents disclosed herein form a crystal clear firm gel that can be used as a base for a cosmetic composition.

As used herein an ester, refers to the group —OC(O)(C1-C6 alkyl).

A “volatile” solvent is a compound capable of evaporating from the skin or lips in less than an hour. Volatile solvents include hydrocarbons such as isododecane. The expression “volatile solvent” or “volatile oil” refers to any non-aqueous medium capable of evaporating on contact with the skin or the lips in less than one hour at room temperature and atmospheric pressure. An aspect of the present disclosure includes one or more volatile solvents chosen from organic solvents, such as volatile cosmetic oils that are liquid at room temperature and have a non-zero vapor pressure, at room temperature and atmospheric pressure, ranging from 10-2 to 300 mm Hg (1.33 to 40,000 Pa), such as greater than 0.03 mmHg (4 Pa), and further such as greater than 0.3 mmHg (40 Pa). The expression “non-volatile oil” as used herein refers to oil that remains on the skin or the lips at room temperature and atmospheric pressure for at least several hours, such as those having a vapor pressure of less than 10-2 mmHg (1.33 Pa).

As used herein, the hydrocarbon-based oil refers to an oil comprising carbon and hydrogen atoms, optionally with at least one group chosen from hydroxyl, ester, carboxyl, or ether groups.

Examples of oils tested are found in Table 1 below.

TABLE 1
Testing of 5% w/w 1-[1-Methyl-2-(1-methyl-2-tridecyloxy-ethoxy)-
ethyl]-3-(6-{3-[1-methyl-2-(1-methyl-2-tridecyloxy-ethoxy)-ethyl]-
ureido}-hexyl)-urea with various oils.

| item | Chemical name | Clarity | Characteristics |
|---|---|---|---|
| 1 | isohexadecane | clear | firm gel |
| 2 | isododecane | clear | firm gel |
| 3 | hydrogenated polyisodecene | clear | crystal clear firm gel |
| 4 | dodecane | clear | crystal clear firm gel |
| 5 | isononyl isononanoate | clear | crystal clear firm gel |
| 6 | C12-15 alkyl benzoate | clear | crystal clear firm gel |
| 7 | polyglyceryl-2 diisostearate | hazy | hazy soft gel. Form the |
| | | | gel after overnight |
| 8 | tridecyl trimellitate | clear | crystal clear firm gel, |
| | | | but softer than lighter |
| | | | oils |
| 9 | diisostearyl malate | haze | hazy soft gel. Form the |
| | | | gel after several hours |
| 10 | hydrogenated polyisodecene | clear | crystal clear firm gel |
| 11 | hydrogenated polyisodecene | haze | hazy soft gel |
| 12 | hydrogenated polyisodecene | clear | clear firm gel with |
| | | | slightly hazy |
| 13 | C12-15 alkyl benzoate/diphenyl | haze | hazy firm gel, but |
| | dimethicone | | softer than |
| | | | TN alone |
| 14 | C12-15 alkyl benzoate/diphenyl | haze | translucent firm gel |
| | dimethicone | | |
| 15 | Butyl stearate | slightly | translucent gel |
| | | haze | |
| 16 | Butyl oleate | clear | clear firm gel |
| 17 | isopropyl palmitate | clear | crystal clear firm gel |
| 18 | isopropyl myristate | clear | crystal clear firm gel |
| 19 | trisiloxane | n/a | not compatible |
| 20 | PPG-3 benzyl ether myristate | hazy | translucent hard gel |
| 21 | octyl palmitate | clear | crystal clear firm gel |
| 22 | isocetyl stearate | slightly | translucent gel |
| | | haze | |
| 23 | cetearyl octanoate | clear | clear firm gel |
| 24 | linseed oil | clear | clear firm gel |
| 25 | soybean oil | clear | clear firm gel |
| 26 | castor oil | slightly | translucent soft gel |
| | | haze | |
| 27 | octyldodecyl neopentanoate | clear | crystal clear firm gel |
| 28 | mineral oil | clear | crystal clear firm gel |

The term stickiness refers to the negative peak force of the compression as measured by a texture analyzer (TA.XT2i Texture Analyzer (Texture Technologies Corp., Scarsdale, N.Y.).

The term flexibility refers to the amount of work returned by the sample as compressive strain is removed to the work required for compression, measurement of the elastic recovery of the sample as measured by the texture analyzer.

The term hardness refers to the peak force of the compression as measured by the texture analyzer.

The texture analyzer is an instrument that applies controlled forces to a cosmetic composition and records a measureable response (deformation, recovery, and time) to the applied force and translates that response into values for stickiness, flexibility, and hardness of the cosmetic composition. Stickiness, flexibility, and hardness are properties that translate to human sensory perception of cosmetic compositions.

The following examples are merely illustrative of the scope of the present invention and are not intended as a limitation upon the scope thereof.

Various tests have been carried out to compare the gelling agents of this invention to traditional gelling agents.

In a first set of experiments a solid gelling agent, without any additional oil pigment, or other material was melted at about 100° C. for each base until all solid material turned liquid. The liquid was poured into a mold and cooled to room temperature.

In a second set of experiments a solid gelling agent was melted at about 100° C. for each base until all solid material turned liquid and then mixed with at least one oil. The liquid was poured into a mold and cooled to room temperature.

### Example 1

### Lipstick Made with Oil and Wax as the Gelling Agent

| | 905- | 905- | 905- | 905- | |
|---|---|---|---|---|---|
| Raw Material | 132-1 | 132-2 | 132-3 | 132-4 | 905-132-5 |
| C12-15 alkyl benzoate | 11.18 | 11.56 | 10.92 | 11.82 | 12.21 |
| isononyl isononanoate | 15.55 | 16.08 | 15.19 | 16.44 | 16.99 |
| hydrogenated | 7.27 | 7.52 | 7.10 | 7.69 | 7.94 |
| polyisobutene | | | | | |
| polybutene | 4.55 | 4.71 | 4.45 | 4.81 | 4.97 |
| hydrogenated | 8.00 | 8.27 | 7.82 | 8.46 | 8.74 |
| polyisobutene | | | | | |
| hydrogenated | 23.45 | 24.25 | 22.91 | 24.79 | 25.62 |
| polyisobutene | | | | | |
| TRIBEHENIN | 4.00 | 3.40 | 4.40 | 3.00 | 2.38 |
| CARNAUBA WAX | 7.00 | 5.95 | 7.70 | 5.25 | 4.17 |
| SYNTHETIC WAX | 5.00 | 4.25 | 5.50 | 3.75 | 2.98 |
| | 16.00 | 13.60 | 17.60 | 12.00 | 9.52 |
| Red lake | 8.00 | 8.00 | 8.00 | 8.00 | 8.00 |
| MICA, TITANIUM | 1.20 | 1.20 | 1.20 | 1.20 | 1.20 |
| DIOXIDE | | | | | |
| TITANIUM | 2.80 | 2.80 | 2.80 | 2.80 | 2.80 |
| DIOXIDE, | | | | | |
| MICA, SILICA | | | | | |
| MICA | 2.00 | 2.00 | 2.00 | 2.00 | 2.00 |
| TOTALS: | 100.00 | 100.00 | 100.00 | 100.00 | 100.00 |

### Example 2

### Lipstick Made with Oil and MT800 as the Gelling Agent

| | 905- | 905- | 905- | 905- | 905- |
|---|---|---|---|---|---|
| | 144-1 | 144-2 | 144-3 | 144-4 | 144-5 |
| Raw Material | 2/14/12 | 2/14/12 | 2/14/12 | 2/14/12 | 2/14/12 |
| C12-15 alkyl benzoate | 12.30 | 12.17 | 12.04 | 12.49 | 11.34 |
| isononyl isononanoate | 17.10 | 16.92 | 16.74 | 17.37 | 15.77 |
| hydrogenated | 8.00 | 7.92 | 7.83 | 8.12 | 7.38 |
| polyisobutene | | | | | |
| polybutene | 5.00 | 4.95 | 4.90 | 5.08 | 4.61 |
| hydrogenated | 8.80 | 8.71 | 8.62 | 8.94 | 8.11 |
| polyisobutene | | | | | |
| hydrogenated | 25.80 | 25.53 | 25.26 | 26.20 | 23.79 |
| polyisobutene | | | | | |
| MT800 | 9.00 | 9.80 | 10.60 | 7.80 | 15.00 |
| Red lake | 8.00 | 8.00 | 8.00 | 8.00 | 8.00 |
| MICA, TITANIUM | 1.20 | 1.20 | 1.20 | 1.20 | 1.20 |
| DIOXIDE | | | | | |
| TITANIUM DIOXIDE, | 2.80 | 2.80 | 2.80 | 2.80 | 2.80 |
| MICA, SILICA | | | | | |
| MICA | 2.00 | 2.00 | 2.00 | 2.00 | 2.00 |
| TOTALS: | 177.00 | 176.20 | 175.40 | 178.20 | 171.00 |
| Ratio oil/gelling | 8.56 | 7.78 | 7.11 | 10.03 | 4.73 |
| agent | | | | | |

The mechanical properties of gelling agent alone and gelling agent mixed with at least one oil were measured by a texture analyzer (TAXT2i Texture Analyzer (Texture Technologies Corp., Scarsdale, N.Y.)

Probe—¼″ spherical stainless steel

Mode—measure force in compression

Option—return to start

Pre-test speed—1 mm/sec

Test speed—1 mm/sec

Post-test speed—1 mm/sec

Trigger force—5 g

Distance—0.5 mm

Tare Mode—Auto

Data Acquisition Rate—500 pps

Data Collected and Processed

Texture Exceed Expert (TEE) Software Version 4 (Texture Technologies Corp.)

Measurements were calculated for hardness, stickiness, and flexibility using the Texture Analyzer.

| Gelling | Hardness | Stickiness | Flexibility |
|---|---|---|---|
| Agent | (g) | (g) | (%) |
| MT800 | 3203.21 | −389.37 | 42.56 |
| Wax* | 5892.06 | −0.33 | 32.62 |
| MT800 + oil | 73.84 | −2.08 | 61.45 |
| Wax* + oil | 175.30 | −27.24 | 16.57 |

*Wax is a composition of Tribehenin/Carnauba/Synthetic Wax in a ratio of 4/7/5

### Example 3

### Antiperspirant/Deodorant Made with MT800 as the Gelling Agent

| RM # | CTFA Names | % | % | % | % |
|---|---|---|---|---|---|
| 110634- | Cyclopentasiloxane | 33.30 | 33.30 | 33.30 | 0.00 |
| 00 | | | | | |
| 098426- | C12-15 ALKYL | 10.00 | 10.00 | 10.00 | 0.00 |
| 01 | BENZOATE | | | | |
| 111041- | DIMETHICONE | 5.00 | 5.00 | 5.00 | 0.00 |
| 00 | | | | | |
| 118098- | Polydecene | 0.00 | 0.00 | 0.00 | 62.50 |
| 00 | | | | | |
| 092579- | Hydrogenated Castor Oil | 4.00 | 7.00 | 0.00 | 0.00 |
| 00 | | | | | |
| 110749- | PEG-8 DISTEARATE | 1.00 | 1.00 | 1.00 | 0.00 |
| 00 | | | | | |
| | | 53.30 | 55.30 | 49.30 | 62.50 |
| 111660- | STEARYL ALCOHOL | 14.00 | 14.00 | 14.00 | 0.00 |
| 00 | | | | | |
| | MT-800 | 4.00 | 1.00 | 8.00 | 12.00 |
| | | 18.00 | 15.00 | 22.00 | 12.00 |
| 113540- | Aluminum Zirconium | 25.50 | 25.50 | 25.50 | 25.50 |
| 00 | Tetrachlorohydrex Gly | | | | |
| 114565- | TALC | 3.00 | 3.00 | 3.00 | 0.00 |
| 00 | | | | | |
| 117555- | ALOE BARBADENSIS | 0.10 | 0.10 | 0.10 | 0.00 |
| 00 | LEAF EXTRACT | | | | |
| 098330- | TOCOPHERYL | 0.10 | 0.10 | 0.10 | 0.00 |
| 01 | ACETATE | | | | |
| | TOTALS: | 171.30 | 170.30 | 171.30 | 174.50 |
| | Ratio of oil to gelling | 3.0 | 3.7 | 2.2 | 5.2 |
| | agent | | | | |

What is claimed is:

1. A composition comprising a gelling agent having a structure of (I): 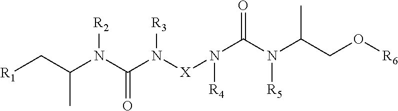
R₂, R₃, R₄, R₅, which are the same or different are selected from a hydrogen, a C₁-C₂₄ alkyl group;
X is selected from a heteroatom, C₂ to C₂₄ alkyl;
R₁ and R₆ which are the same or different are selected from a hydrogen, a C₁-C₂₄ alkyl group, a heteroatom;
and an at least one oil, wherein the oil and gelling agent are in a ratio of about 2 to about 16.

2. The composition of claim 1, wherein the oil to gelling agent ratio is about 4 to about 10.

3. The composition of claim one, wherein the gelling agent is 1-[1-Methyl-2-(1-methyl-2-tridecyloxy-ethoxy)-ethyl]-3-(6-{3-[1-methyl-2-(1-methyl-2-tridecyloxy-ethoxy)-ethyl]-ureido}-hexyl)-urea.

4. The composition of claim 1, wherein the composition is a cosmetic composition.

5. The composition of claim 1, wherein the composition is anhydrous.

6. The composition of claim 1, further comprising at least one additive chosen from an antioxidant, a filler, a film-former, a preserving agent, a fragrance, a neutralizing agent, a thickener, a cosmetic composition active agent, an emollient, and mixtures thereof.

7. The composition of claim 1, wherein the composition further comprises an at least one pigment, dye, or stain.

8. The composition of claim 1, wherein the gelling agent is present in a concentration of about 6 to 14 weight percent of the composition.

9. The composition of claim 1, further comprising at least one solvent.

10. The composition of claim 1, wherein the composition is a clear gel.

11. The composition of claim 1, wherein the composition is a stick.

12. The composition of claim 1, wherein the composition has a flexibility of >about 30%.

13. The composition of claim 1, wherein the composition has a stickiness of <about −10 grams.

14. The composition of claim 1, wherein the composition has a hardness of <about 100 grams.

15. The composition of claim 1, wherein the composition is a water-in-oil emulsion.

16. The composition of claim 15, wherein the water to oil ratio is about 0.1 to about 80.

17. The composition of claim 1, wherein the composition is a stick having a pigmented core surrounded by a clear outer shell.

18. The composition of claim 15, wherein the water phase and oil phase have a matched refractive index.

19. The composition of claim 4, wherein the cosmetic composition is an antiperspirant/deodorant.
